(19) United States
(12) Patent Application Publication
(10) Pub. No.: US 2008/0125490 A1
(43) Pub. Date: May 29, 2008

(54) Treatment and prevention of cardiovascular disease in patients with chronic kidney disease by administering Omega-3 Fatty Acids

(76) Inventors: My Svensson, Vadum (DK); Jeppe Hagstrup Christensen, Aalborg (DK); Erik Berg Schmidt, Skerping (DK); Kaj Anker Jorgensen, Langa (DK)

Correspondence Address: ARENT FOX LLP, 1050 CONNECTICUT AVENUE, N.W., SUITE 400, WASHINGTON, DC, 20036, US

(21) Appl. No.: 11/979,539

(22) Filed: Nov. 5, 2007

Related U.S. Application Data
(60) Provisional application No. 60/856,299, filed on Nov. 3, 2006.

(51) Int. Cl. A61K 31/202; A61P 9/00

(52) U.S. Cl. 514/560

(57) ABSTRACT

Compositions comprising omega-3 fatty acids are provided, where the compositions are useful for treating cardiovascular disease in patients suffering from chronic kidney disease (CKD), preventing its further progression, and treating underlying risk factors such as hypertension, dyslipidemia, obesity and/or diabetes. Also provided are methods of using the compositions to reduce the occurrence of or prevent major coronary events, including myocardial infarctions, in patients with CKD.

### RELATED APPLICATION DATA

This application claims priority from U.S. Provisional Application No. 60/856,299, which was filed on Nov. 3, 2006, the contents of which are incorporated herein by reference.

### BACKGROUND OF THE INVENTION

1. Field of the Invention

The present invention relates, generally, to compositions comprising omega-3 fatty acids, where the compositions are useful for treating, reducing the occurrence of, and/or preventing cardiovascular disease in patients suffering from chronic kidney disease. The present invention also includes pharmaceutical formulations made from the compositions, methods of using the formulations to treat, reduce the occurrence of, or prevent cardiovascular disease in patients suffering from chronic kidney disease, and methods of using the formulations to treat patients with chronic kidney disease who are also suffering from any of the various underlying conditions that may lead to major coronary events, including hypertension, dyslipidemia, obesity and/or diabetes. Preferably, the compositions may be used to reduce the occurrence of or prevent major coronary events in patients with CKD.

2. Description of the Related Art

Chronic kidney disease (CKD) is may be characterized as a continuum including chronic renal insufficiency (defined as serum creatinine levels of 1.5-3.0 mg/dL), chronic renal failure (serum creatinine levels above 3.0 mg/dL), and end-stage renal disease. The National Kidney Foundation's Kidney Disease Outcomes Quality Initiative (K/DOQI), available at the website address http://www.kidney.org/professionals/kdoqi/guidelines_ckd/toc.htm and herein incorporated by reference it its entirety, defines five stages of CKD. Almost 20 million people in the United States suffer from CKD. Approximately two-thirds of CKD cases in the U.S. are due to hypertension or diabetes, but CKD can also be caused by glomerulonephritis, polycystic kidney disease, Alport syndrome, reflux nephropathy, obstructive uropathy, kidney stones, infections, and analgesic nephropathy. Early in the disease progression, patients may not experience any symptoms, but as the kidney function continues to decline, symptoms may include lethargy, weakness, loss of appetite, insomnia, inability to think clearly, and edema. As the kidneys stop functioning, patients may experience vomiting, weakness, confusion, and coma. As the disease progresses to end-stage renal disease (ESRD), hemodialysis or renal transplantation are necessary to prevent death.

In addition to the problems associated with reduced kidney function, patients suffering from chronic kidney disease are at increased risk of suffering from cardiovascular disease (CVD) and experiencing major coronary events (MCEs). Such MCEs may include, but are not limited to, myocardial infarction (Ml) and coronary intervention such as coronary revascularization, investigational or interventional angioplasty, percutaneous transluminal coronary angioplasty (PTCA), percutaneous coronary intervention (PCI) and coronary artery bypass graft (CABG). CVD is the most common cause of death in patients with end-stage renal disease. See, e.g., Cheung et al., Kidney Int 65(6):2380-9 (2004). Patients with varying degrees of CKD who are not yet dialysis-dependent also have an increased risk of morbidity and mortality from coronary artery disease.

Patients with CKD often exhibit traditional risk factors for cardiovascular disease, such as diabetes, low serum HDL cholesterol levels, hypertension, left ventricular hypotrophy, and increased age. Many CKD patients exhibit two or more of these risk factors. Due to their elevated risk of cardiovascular disease, patients with chronic kidney disease should be monitored closely to identify any existing cardiovascular risk factors, as well as to treat known risk factors for cardiovascular disease.

In the general population, dyslipidemia is an important risk factor used to predict the likelihood of developing CVD. In humans, cholesterol and triglycerides are part of lipoprotein complexes in the bloodstream, and can be separated via ultracentrifugation into high-density lipoprotein (HDL), intermediate-density lipoprotein (IDL), low-density lipoprotein (LDL) and very-low-density lipoprotein (VLDL) fractions. Cholesterol and triglycerides are synthesized in the liver, incorporated into VLDL, and released into the plasma. High levels of total cholesterol (total-C), LDL-C, and apolipoprotein B (a membrane complex for LDL-C) promote human atherosclerosis and decreased levels of HDL-C and its transport complex, apolipoprotein A, which are associated with the development of atherosclerosis. Further, cardiovascular morbidity and mortality in humans can vary directly with the level of total-C and LDL-C and inversely with the level of HDL-C.

However, unlike the general population, the relationship between lipid-associated atherosclerosis and cardiovascular outcomes in CKD patients is quite distinct. Holdaas et al., Am. J. Cardiovasc. Drugs 5(4):255-269 (2005), reviewed prior studies of CKD patients in which traditional hyperlipidemia therapies were employed, and noted that the results of these studies were negative. Holdaas et al. argue that clinical trial results from the general population may not be applicable to patients with CKD, since these patients exhibit additional non-traditional risk factors for cardiovascular disease, such as reduced glomerular filtration rate, albuminuria/proteinuria, anemia, or electrolyte imbalances that may account for differences in disease outcomes and response to therapy. For example, as cited by Holdaas, dialysis patients display an inverse relationship between atherogenic lipids and cardiovascular outcomes. Furthermore, Holdaas states that for the dialysis patient, there are currently no data for making recommendations as to lipid lowering therapy for preventing primary cardiovascular events.

Omega-3 fatty acids are known to reduce serum triglycerides by inhibiting diacylglycerol acyltransferase (DGAT) and by stimulating peroxisomal and mitochondrial beta oxidation. Marine oils, also commonly referred to as fish oils, are a good source of two omega-3 fatty acids, eicosapentaenoic acid (EPA) and docosahexaenoic acid (DHA), which have been found to regulate lipid metabolism. Omega-3 fatty acids have been found to have beneficial effects on the risk factors for cardiovascular diseases, especially mild hypertension, hypertriglyceridemia and on the coagulation factor VII phospholipid complex activity. Omega-3 fatty acids lower serum triglycerides, increase serum HDL-cholesterol, lower systolic and diastolic blood pressure and the pulse rate, and lower the activity of the blood coagulation factor VII-phospholipid complex. Further, omega-3 fatty acids seem to be well tolerated, without giving rise to any severe side effects.

One form of omega-3 fatty acid is a concentrate of omega-3, long chain, polyunsaturated fatty acids from fish oil containing DHA and EPA that is sold under the trademark Lovaza™ (formerly sold as Omacor®). Such a form of omega-3 fatty acid is described, for example, in U.S. Pat. Nos. 5,502,077, 5,656,667 and 5,698,594, each of which is incorporated herein by reference.

The use of fish oil compositions to prevent cardiovascular events has been investigated in the general population. PCT Published Application No. WO 00/48592 discloses the use of compositions containing omega-3 fatty acids to prevent and/or reduce cardiovascular events in patients who have experienced a prior myocardial infarction.

Various studies have been conducted to determine the effects of dietary fish intake or dietary supplementation with products such as fish oil on risk factors associated with cardiovascular disease in patients suffering from chronic kidney disease. Although some studies have indicated that fish oil supplementation may provide beneficial effects to chronic kidney disease patients, they have not resulted in specific dietary supplementation guidelines or disclosed methods of treating CVD or MCEs with pharmaceutical compositions of fish oil or pharmaceutical compositions of omega-3 fatty acids or derivatives.

Kutner et al., Am. J. Kidney Diseases (May 2002) Vol. 39, No. 5, pp. 1018-1024, analyzed patient survival with dietary fish consumption in patients with chronic renal failure. It was found that patients who died over the course of the three-year study were less likely to eat fish.

Rylance et al., Nephron (1986) Vol. 43, No. 3, pp. 196-202, studied the effects of fish oil on lipid and platelet function in hemodialysis patients, and found that consumption of fish oil was associated with significantly beneficial effects on blood lipids and reduced blood pressure levels.

Toto et al., Blood Purification (1996) Vol. 14, No. 1, pp. 75-82, observed that patients with chronic renal failure and ESRD commonly exhibit dyslipidemia, and recommended further study of the effects of treatment with medications such as fibric acid, nicotinic acid, statins, and/or fish oil.

Svensson et al., Am. J. Kidney Diseases (2004) Vol. 44, No. 1, pp. 77-83, conducted a study to examine the effect of an eight-week regimen of n-3 PUFAs on plasma lipid levels, lipoprotein levels, and 24-hour ambulatory blood pressure in patients with chronic renal failure. No effect was found on blood pressure, but there was a significant increase in HDL cholesterol, and a significant decrease in serum triglyceride levels.

Christensen et al., Clin. Nephrol. (1998) Vol. 49, No. 2, pp. 102-106, conducted a study to examine the 24-hour heart rate variability in patients with chronic renal failure (CRF), as compared to patients having a history of myocardial infarction. n-3 PUFA content in cell membranes was also measured in the CRF patients. A twelve-week dietary intervention with 5.2 grams n-3 PUFAs/day was conducted, after which the heart rate variability and n-3 PUFA content in the cell membranes were measured again. The intervention revealed a positive association between higher heart rate variability and higher n-3 PUFA content in cell membranes.

Phinney et al., U.S. Patent Publication No. 2005/0137253, discloses that treatment with compositions comprising non-alpha tocopherol and omega-3 fatty acids such as DHA, are useful for treating inflammatory conditions.

None of the above-mentioned approaches provides compositions and methods for treating, reducing the occurrence of and/or preventing cardiovascular disease in patients suffering from chronic kidney disease, or methods of treating underlying risk factors (such as hypertension, dyslipidemia, obesity and/or diabetes) that are associated with development of cardiovascular disease, especially in patients with chronic kidney disease, by administering the compositions of the present invention containing omega-3 fatty acids, and optionally including one or more additional compounds useful in treatment or prevention of cardiovascular disease. In addition, none of the above-mentioned studies provides compositions and methods that may be used to reduce the occurrence of or prevent major coronary events (MCEs), such as myocardial infarctions (MIs), in patients with CKD.

There is clearly a great need in the art for compositions that are useful for treating, reducing the occurrence of, and/or preventing CVD in patients with CKD. Methods of reducing the occurrence of or preventing MCEs using the formulations are especially needed. There is also a need in the art for additional treatments effective in alleviating the underlying conditions associated with cardiovascular disease.

### SUMMARY OF THE INVENTION

The present invention meets the unmet needs of the art, as well as others, by providing compositions containing omega-3 fatty acids that preferably include EPA and DHA, preferably Lovaza™ omega-3 fatty acids, that can provide an effective treatment for CVD, or reduce or prevent its occurrence, as well as treat any of the various underlying conditions that may lead to the development of CVD. These additional underlying conditions may include, but are not limited to, hypertension, dyslipidemia, obesity and/or diabetes. According to a particularly preferred embodiment, the underlying condition is dyslipidemia. According to a further embodiment, the omega-3 fatty acids are provided for co-administration, or as unit doses, with one or more compounds useful for treating CVD, or the underlying conditions responsible for causing CVD, in CKD patients.

The present invention also provides natural or synthetic omega-3 fatty acids, and/or their pharmaceutically acceptable esters, derivatives, conjugates, precursors or salts, or mixtures thereof, to provide an effective pharmaceutical treatment for CVD in CKD patients, while minimizing unwanted side effects.

One embodiment of the present invention provides a method of utilizing a composition comprising natural or synthetic omega-3 fatty acids, and/or their pharmaceutically acceptable esters, derivatives, conjugates, precursors or salts, or mixtures thereof, in reducing the occurrence of or the prevention of CVD in CKD patients. According to a preferred embodiment, the composition may be utilized to reduce the occurrence of or prevent major coronary events, such as myocardial infarctions, in patients with CKD.

Another embodiment of the present invention is a oral formulation of natural or synthetic omega-3 fatty acids or pharmaceutically acceptable esters, derivatives, conjugates, precursors or salts thereof, or mixtures thereof, in the treatment of one or more risk factors for CVD exhibited by a CKD patient. In one aspect of the embodiment, the risk factors may include hypertension, dyslipidemia, obesity and/or diabetes.

Another subject of the invention is a method of treating, reducing the occurrence of, and/or preventing CVD in a patient suffering from CKD, by providing a composition comprising natural or synthetic omega-3 fatty acids or pharmaceutically acceptable esters, derivatives, conjugates, precursors or salts thereof, or mixtures thereof, and thereafter administering the composition to the patient.

Another subject of the invention is the use of natural or synthetic omega-3 fatty acids or pharmaceutically acceptable esters, derivatives, conjugates, precursors or salts thereof, or mixtures thereof, for the manufacture of a medicament for treating CVD in CKD patients.

The compositions and methods of the present invention may further comprise co-administration of one or more additional compounds useful in the treatment of CVD or one or more of hypertension, dyslipidemia, obesity and/or diabetes. According to a particularly preferred embodiment, the compositions and methods of the present invention are useful in the treatment of dyslipidemia. Also included are unit dosage forms including the omega-3 fatty acids and said one or more additional compounds, and methods for administering same to a patient in need thereof.

In preferred embodiments the pharmaceutical compositions comprise Lovaza™ omega-3 fatty acids, as described in U.S. Pat. Nos. 5,502,077, 5,656,667 and 5,698,594. In other preferred embodiments the pharmaceutical compositions comprise omega-3 fatty acids present in a concentration of at least 40% by weight as compared to the total fatty acid content of the composition. In still other preferred embodiments the omega-3 fatty acids comprise at least 50% by weight of EPA and DHA as compared to the total fatty acid content of the composition, and the EPA and DHA are in a weight ratio of EPA:DHA of from 99:1 to 1:99, preferably from 1:4 to 4:1, more preferably from 1:3 to 3:1, and most preferably from 1:2 to 2:1.

Other novel features and advantages of the present invention will become apparent to those skilled in the art upon examination of the following or upon learning by practice of the invention.

### DETAILED DESCRIPTION OF THE INVENTION

The present invention relates to compositions comprising omega-3 fatty acids, methods of making same, and their use in treating, reducing the occurrence of, and/or preventing CVD in patients suffering from CKD, as well as their use in treating the underlying risk factors that are associated with CVD. The terms “cardiovascular disease” or “CVD” include major coronary events (MCEs) which include, but are not limited to, myocardial infarction (Ml) and coronary intervention such as coronary revascularization, investigational or interventional angioplasty, percutaneous transluminal coronary angioplasty (PTCA), percutaneous coronary intervention (PCI) and coronary artery bypass graft (CABG); and CVD also includes angina pectoris, documented coronary atherosclerosis, stroke, transient ischemic attack (TIA) and peripheral artery disease (PAD). Major adverse cardiovascular events (MACE) include cardiac death, other cardiovascular death, MCE, hospitalization for unstable angina, stroke, TIA and hospitalization for PAD. Risk factors for CVD in patients with CKD include hypertension, dyslipidemia, obesity and/or diabetes. Additional compounds useful in treating, reducing the occurrence of, or preventing CVD or the underlying risk factors associated with CVD may also be beneficially coadministered with the inventive compositions and pharmaceutical formulations, or may be provided in a unit dose form therewith. According to a particularly preferred embodiment, the compositions and methods of the present invention are useful in treating, reducing the occurrence of, and/or preventing MCEs.

### 1. Compositions Containing Omega-3 Fatty Acids

In preferred embodiments, the compositions of the present invention are useful for treating cardiovascular disease, and the underlying cause(s) thereof. The compositions are preferably useful for reducing the occurrence of or preventing major coronary events such as myocardial infarction (Ml) and coronary intervention such as coronary revascularization, angioplasty, percutaneous transluminal coronary angioplasty (PTCA), percutaneous coronary intervention (PCI) and coronary artery bypass graft (CABG). These inventive compositions preferably comprise Lovaza™ omega-3 fatty acids, as described in U.S. Pat. Nos. 5,502,077, 5,656,667 and 5,698,594. In other preferred embodiments the compositions comprise omega-3 fatty acids present in a concentration of at least 40% by weight as compared to the total fatty acid content of the composition. In still other preferred embodiments the omega-3 fatty acids comprise at least 50% by weight of EPA and DHA as compared to the total fatty acid content of the composition, and the EPA and DHA are in a weight ratio of EPA:DHA of from 99:1 to 1:99, preferably from 1:4 to 4:1, more preferably from 1:3 to 3:1, and most preferably from 1:2 to 2:1. The omega-3 fatty acids may comprise pure EPA or pure DHA.

As used herein, the term “omega-3 fatty acids” includes natural or synthetic omega-3 fatty acids, or pharmaceutically acceptable esters, derivatives, conjugates (see, e.g., Zaloga et al., U.S. Patent Application Publication No. 2004/0254357, and Horrobin et al., U.S. Pat. No. 6,245,811, each hereby incorporated by reference), precursors or salts thereof and mixtures thereof. Examples of omega-3 fatty acid oils include but are not limited to omega-3 polyunsaturated, long-chain fatty acids such as a eicosapentaenoic acid (EPA), docosahexaenoic acid (DHA), and a-linolenic acid; esters of omega-3 fatty acids with glycerol such as mono-, di- and triglycerides; and esters of the omega-3 fatty acids and a primary, secondary or tertiary alcohol such as fatty acid methyl esters and fatty acid ethyl esters. Preferred omega-3 fatty acid oils are long-chain fatty acids such as EPA or DHA, triglycerides thereof, ethyl esters thereof and mixtures thereof. The omega-3 fatty acids or their esters, derivatives, conjugates, precursors, salts and mixtures thereof can be used either in their pure form or as a component of an oil such as fish oil, preferably purified fish oil concentrates. Commercial examples of omega-3 fatty acids suitable for use in the invention include Incromega F2250, F2628, E2251, F2573, TG2162, TG2779, TG2928, TG3525 and E5015 (Croda International PLC, Yorkshire, England), and EPAX6000FA, EPAX5000TG, EPAX4510TG, EPAX2050TG, K85TG, K85EE, K80EE and EPAX7010EE (EPAX a.s., 1327 Lysaker, Norway).

Another preferred composition includes omega-3 fatty acids present in a concentration of at least 40% by weight, preferably at least 50% by weight, more preferably at least 60% by weight, still more preferably at least 70% by weight, most preferably at least 80% by weight, or even at least 90% by weight. Preferably, the omega-3 fatty acids comprise at least 50% by weight of EPA and DHA, more preferably at least 60% by weight, still more preferably at least 70% by weight, most preferably at least 80%, such as about 84% by weight. Preferably the omega-3 fatty acids comprise about 5 to about 100% by weight, more preferably about 25 to about 75% by weight, still more preferably about 40 to about 55% by weight, and most preferably about 46% by weight of EPA. Preferably the omega-3 fatty acids comprise about 5 to about 100% by weight, more preferably about 25 to about 75% by weight, still more preferably about 30 to about 60% by weight, and most preferably about 38% by weight of DHA. All percentages above are by weight as compared to the total fatty acid content in the composition, unless otherwise indicated. The percentage by weight may be based on the free acid or ester forms, although it is preferably based on the ethyl ester form of the omega-3 fatty acids even if other forms are utilized in accordance with the present invention.

The omega-3 fatty acids can be present in an amount from about 350 mg to about 10 grams, more preferably about 500 mg to about 6 grams, and most preferably from about 750 mg to about 4 grams. A particularly preferred amount of omega-3 fatty acids is about 2 grams. This amount may be in one or more dosage forms, preferably one dosage form. The omega-3 fatty acid composition optionally includes chemical antioxidants, such as alpha tocopherol, oils, such as soybean oil and partially hydrogenated vegetable oil, and lubricants such as fractionated coconut oil, lecithin and a mixture of the same.

The most preferred form of omega-3 fatty acids is Lovaza™ (omega-3 acid ethyl esters) (K85EE, Pronova BioPharma A.S., Lysaker, Norway) and preferably comprises the following characteristics (per dosage form):

| Test | Minimum Value | Maximum Value |
|---|---|---|
| Eicosapentaenoic acid C20:5 EE | 430 mg/g | 495 mg/g |
| Docosahexaenoic acid C22:6 EE | 347 mg/g | 403 mg/g |
| EPA-EE and DHA-EE | 800 mg/g | 880 mg/g |
| Total n-3 fatty acids | 90% (w/w) | |

The active ingredient of the present invention, omega-3 fatty acids, may be administered with a combination of one or more non-active pharmaceutical ingredients (also known generally herein as “excipients”). Non-active ingredients, for example, serve to solubilize, suspend, thicken, dilute, emulsify, stabilize, preserve, protect, color, flavor, and fashion the active ingredients into an applicable and efficacious preparation that is safe, convenient, and otherwise acceptable for use.

The omega-3 fatty acids may optionally be co-administered with one or more additional compounds, or provided in a unit dose pharmaceutical formulation with one or more additional compounds, where those additional compounds are useful in reducing the occurrence of or preventing CVD from occurring or progressing, are useful in reducing the occurrence of or preventing MCEs, or are effective in treating any of the underlying risk factors that are commonly associated with CVD.

The additional compounds in accordance with the present invention may be selected from the group consisting of angiotensin-converting-enzyme (ACE) inhibitors; angiotensin receptor blockers (ARBs); renin inhibitors; dyslipidemic agents such as HMG CoA reductase inhibitors (statins) (preferably including, but not limited to, pitavastatin, simvastatin, rosuvostatin, pravastatin, atorvastatin, lovastatin, and fluvastatin); dihydropyridine calcium channel blockers (preferably including, but not limited to, Bay K 8644, amlodipine, felodipine, lacidipine, lercanidipine, nicardipine, nifedipine, nimodipine, nisoldipine, nitrendipine and isradipine; antiarrhythmic agents (preferably including, but not limited to, quinidine, procainamide, disopyramide, lidocaine, mexiletine, tocainide, phenytoin, encainide, flecainide, moricizine, propafenone, esmolol, propranolol, acebutolol, metoprolol, amiodarone, azimilide, bretylium, clofilium, dofetilide, ibutilide, sematilide, sotalol, verapamil, mebefradfil, diltiazem adenosine, and digoxin); azetidinone-based cholesterol absorption inhibitors (preferably including, but not limited to, ezetimibe, MD-0727, and SCH60663); niacin and derivatives (preferably including, but not limited to, nicotinamide); PPAR agonists/antagonists (preferably including, but not limited to, PPAR-alpha, PPAR-gamma, PPAR-delta, PPAR-alpha/gamma, PPAR-gamma/delta, PPAR-alpha/delta, and PPAR-alpha/gamma/delta agonists and antagonists, as well as partial agonists and/or antagonists, including but not limited to fibrates such as fenofibrate, tesaglitazar, naviglitizar, mivaglitazar and thiazolidinediones such as pioglitazone and rosiglitazone); bile acid sequestrants (preferably including, but not limited to, cholestyramine, cholestipol, and colesevelam); antiplatelet drugs (including, but not limited to, aspirin, clopidogrel, and ticlopidine); and pharmaceutically acceptable esters, derivatives, conjugates, precursors or salts thereof, and mixtures thereof.

Also envisioned in accordance with the present invention is the use of similar compounds to those set forth above, which may be discovered in the future, or already existing compounds that may be approved for new uses in the future. Further envisioned is the optional inclusion of additional compounds useful in the treatment of conditions such as hypertension and diabetes, which are known to contribute to cardiovascular disorders.

Where provided, these optional additional active ingredients are useful in the treatment of cardiovascular disease, or any of the underlying risk factors or diseases that cause CVD. These may include, but are not limited to, hypertension, dyslipidemia, obesity and/or diabetes.

The optional additional active ingredients, when provided, are including in amounts that are sufficient to treat, reduce the occurrence of and/or prevent CVD, reduce the occurrence of or prevent MCEs, and/or treat the underlying risk factors that are elevating the risk of CVD in a patient suffering from CKD. The optional additional ingredients are provided in amounts that are generally regarded as safe, and are effective in treating, reducing the occurrence of, and/or preventing CVD and treating its underlying causes.

The composition comprising concentrated omega-3 fatty acids may be prepared in the form of a capsule, such as a hard gelatin capsule; a tablet; a powder that can be dispersed in a beverage; a liquid; or a soft gel capsule. The composition may also be contained in a liquid suitable for injection or infusion. However, the methods of preparing the inventive compositions for administration are not to be limited to any particular dosage form. Rather, they may be prepared as any pharmaceutically acceptable dosage form, including other solid oral dosage forms, other liquid oral dosage forms, and any other suitable dosage forms. When provided, the one or more optional additional active ingredients may also be provided in the dosage form, as a homogeneous solution or a heterogenous suspension with a solvent comprising the omega-3 fatty acids, so as to create a convenient unit dose form, or a soft gelatin capsule coated with the one or more optional additional active ingredients (see U.S. Provisional Patent Application No. 60/780,306, hereby incorporated by reference).

In some embodiments, the unit dose formulations of the present invention allow for improved effectiveness of each active ingredient, with one or both administered as a conventional full-strength dose, as compared to the formulations in the prior art. In other embodiments, the formulations of the present invention may allow for reduced dosages of the optional additional ingredients, such as 25-80% of the conventional full-strength dose, as compared to the formulations in the prior art, while still maintaining or even improving upon the effectiveness of each active ingredient.

The present combinations of concentrated omega-3 fatty acids and one or more additional ingredients, taken from the list set forth above, may allow for a greater effect than any expected combined or additive effect of the compounds alone. Thus, the combined treatment using the active ingredients, separately or through the novel combination product of the present invention, may cause an unexpected increase in effect of the active ingredients. This may allow increased effectiveness with standard dosages, or, alternatively, may allow maintained effectiveness with reduced dosages of the active ingredients.

### 2. Methods of Treating, Reducing the Occurrence of and/or Preventing Cardiovascular Disease in Patients Suffering from Chronic Kidney Disease

The compositions containing omega-3 fatty acids described above can be administered in a daily amount of from about 0.1 g to about 10 g, more preferably about 1 g to about 6 g, and most preferably from about 2 g to about 4 g, to a patient suffering from CKD in order to treat, reduce the occurrence of, and/or prevent CVD from developing or progressing. Administration of about 2 g/day of the composition, containing about 1.7 g of omega-3 fatty acids, is particularly preferred. The daily dosages of concentrated omega-3 fatty acids can be administered together in from 1 to 10 dosages, with the preferred number of dosages from 1 to 4 times a day, most preferred 1 to 2 times a day. The administration is preferably oral administration, although other forms of administration that provide a unit dosage of concentrated omega-3 fatty acids may be used. The administration of the dosages is preferably effective in treating, reducing the occurrence of, and/or preventing CVD in a CKD patient, and/or is effective in treating the underlying risk factor(s) responsible for causing the CVD in the patient suffering from CKD, particularly hypertension, dyslipidemia, obesity and/or diabetes.

The compositions containing omega-3 fatty acids can optionally be co-administered with one or more additional compounds useful for alleviating CVD in a CKD patient, and/or is effective in treating the underlying risk factor(s) responsible for causing the CVD in the patient suffering from CKD, particularly hypertension, dyslipidemia, obesity and/or diabetes. Administration of unit dose forms of the omega-3 fatty acids and one or more additional compounds, as listed above, is also contemplated in accordance with the present invention.

In one embodiment, treatment in accordance with the present invention normalizes blood lipid levels and/or reduces systolic blood pressure levels to under 140 mmHg and/or reduces diastolic blood pressure levels to under 90 mmHg. Blood lipid levels may be measured in accordance with any accepted method in the art. Blood pressure levels may be measured in accordance with any recognized method in the art, including, but not limited to, supine, sitting, upright, standing or 24-hr ambulatory.

In a study conducted by Svensson et al., Clin. J. Am. Soc. Nephrol. 1:780-786 (2006), which was submitted as part of U.S. Provisional Application No. 60/856,299, the disclosure of which is hereby incorporated by reference in its entirety, patients with established CVD who had undergone at least six months of stable hemodialysis treatment were assessed for inclusion in a study to determine the effect of fish oil as secondary prevention for CVD. Of 717 patients assessed for eligibility, 206 were selected and randomized into a treatment group (n=103) that received 2 capsules of Lovaza™ (total dose of 1.7 g/day omega-3 fatty acids) daily, and a placebo group (n=103) that received 2 capsules containing olive oil. The patients were followed for two years, during which time 4 patients in the Lovaza™ treatment group experienced MIs, while 13 patients in the control group experienced MIs, which was a significant difference (P=0.036). In addition, 7 patients in the Lovaza™ treatment group experienced a major coronary event, while 17 patients in the control group experienced a major coronary event, which was a significant difference (P=0.043).

It will, of course, be appreciated that the above description has been given by way of example only and that modifications in detail may be made within the scope of the present invention.

Throughout this application, various patents and publications have been cited. The disclosures of these patents and publications in their entireties are hereby incorporated by reference into this application, in order to more fully describe the state of the art to which this invention pertains.

The invention is capable of considerable modification, alteration, and equivalents in form and function, as will occur to those ordinarily skilled in the pertinent arts having the benefit of this disclosure.

While the present invention has been described for what are presently considered the preferred embodiments, the invention is not so limited. To the contrary, the invention is intended to cover various modifications and equivalent arrangements included within the spirit and scope of the detailed description provided above.

What is claimed:

1. A method of reducing the occurrence of or preventing cardiovascular disease (CVD) in a chronic kidney disease (CKD) patient, comprising the steps of:
providing a composition comprising one or more omega-3 fatty acids; and
administering the composition to a CKD patient in an amount effective to reduce the occurrence of or prevent CVD and/or one or more major adverse cardiovascular events.

2. A method of treating CVD in a patient suffering from CKD, comprising the steps of
providing a composition comprising one or more omega-3 fatty acids; and
administering the composition to a CKD patient in an amount effective to treat CVD and/or one or more major adverse cardiovascular events.

3. The method of claim 2, wherein the method provides an effective treatment for underlying conditions or risk factors of major coronary events.

4. The method of claim 1, wherein the method reduces the occurrence of or prevents the occurrence of a major coronary event.

5. The method of claim 4, wherein the major coronary event is selected from the group consisting of myocardial infarction; and coronary intervention selected from the group consisting of coronary revascularization, angioplasty, percutaneous transluminal coronary angioplasty, percutaneous coronary intervention, and coronary artery bypass graft.

6. The method of claim 1, wherein the method reduces the occurrence of or prevents myocardial infarction.

7. The method of claim 1, wherein the method provides an effective treatment for conditions selected from the group consisting of hypertension, dyslipidemia, obesity, and diabetes.

8. The method of claim 1, wherein the one or more omega-3 fatty acids comprise EPA and DHA.

9. The method of claim 1, wherein the one or more omega-3 fatty acids are present in a concentration of at least 40% by weight as compared to the total fatty acid content of the composition.

10. The method of claim 1, wherein the one or more omega-3 fatty acids are present in a concentration of at least 80% by weight as compared to the total fatty acid content of the composition.

11. The method of claim 1, wherein the one or more omega-3 fatty acids comprise at least 80% by weight of EPA and DHA as compared to the total fatty acid content of the composition.

12. The method of claim 1, wherein the one or more omega-3 fatty acids comprise about 40% to about 55% by weight of EPA as compared to the total fatty acid content of the composition.

13. The method of claim 1, wherein the one or more omega-3 fatty acids comprise about 30% to about 60% by weight of DHA as compared to the total fatty acid content of the composition.

14. The method of claim 1, wherein the one or more omega-3 fatty acids are selected from the group consisting of omega-3 polyunsaturated, long-chain fatty acids; esters of omega-3 fatty acids with glycerol; esters of omega-3 fatty acids and a primary, secondary or tertiary alcohol; and mixtures thereof.

15. The method of claim 1, wherein the EPA and DHA are in a weight ratio of EPA:DHA of from 4:1 to 1:4.

16. The method of claim 1, wherein the EPA and DHA are in a weight ratio of EPA:DHA of from 1:2 to 2:1.

17. The method of claim 1, wherein the one or more omega-3 fatty acids are provided for co-administration with one or more additional compounds selected from the group consisting of angiotensin-converting-enzyme inhibitors; angiotensin receptor blockers; renin inhibitors; HMG CoA reductase inhibitors; dihydropyridine calcium channel blockers; antiarrhythmic agents; azetidinone-based cholesterol absorption inhibitors; niacin and derivatives; PPAR agonists/antagonists; bile acid sequestrants; antiplatelet drugs; and pharmaceutically acceptable esters, derivatives, conjugates, precursors or salts thereof.

18. The method of claim 17, wherein the one or more omega-3 fatty acids are administered apart from administration of the one or more additional compounds.

19. The method of claim 17, wherein the one or more omega-3 fatty acids are administered simultaneous to the administration of the one or more additional compounds.

20. The method of claim 17, wherein the one or more omega-3 fatty acids and the one or more additional compounds are administered in a concomitant treatment regime.

21. The method of claim 17, wherein the one or more omega-3 fatty acids and the one or more additional compounds are administered together in a unit dose form.
